(19) United States
(12) Patent Application Publication
(10) Pub. No.: US 2018/0196282 A1
(43) Pub. Date: Jul. 12, 2018

(54) BINOCULAR LOUPES

(71) Applicants: Kikuko YONEZAWA, Higashichikuma-gun, Nagano (JP); MEDSOLEIL CO., LTD., Higashichikuma-gun, Nagano (JP)

(72) Inventor: Kikuko YONEZAWA, Higashichikuma-gun, Nagano (JP)

(21) Appl. No.: 15/740,128

(22) PCT Filed: Jul. 1, 2016
(86) PCT No.: PCT/JP2016/069704

§ 371 Date: Dec. 27, 2017

(30) Foreign Application Priority Data
Jul. 2, 2015 (JP) 2015-133785

(51) Int. Cl. G02C 7/08; G02B 23/18; G02B 25/00; G02C 9/00

(52) U.S. Cl. CPC G02C 7/088; G02B 23/18; G02C 7/086; A61B 2090/502; G02C 9/00; G02C 2200/02; G02B 25/004

(57) ABSTRACT

Provided are binocular loupes capable of ensuring an optimal focal distance at the time of focusing for a user using a simple operation. A focus adjustment unit disposed in the eyepiece units of a pair of left and right loupe bodies of binocular loupes is provided with a focus adjustment lens and a magnet ring. The magnet ring is mounted in contact with a corrective lens constituting an optical system inside the loupe bodies and is secured by a holding ring. The focus adjustment lens has a magnetic body at the peripheral edge drawn to the magnet and is fitted into and held by an annular ring of the magnetic ring. The focal adjustment lens can thereby be detachably mounted on the loupe bodies.

### TECHNICAL FIELD

The present invention relates to binocular loupes used in medical operations and precision work operation.

### BACKGROUND ART

Binocular loupes have conventionally been used widely in each field of medical field, precision work, jewel processing and the like, as means for enlarging a local visual object on hand to visually identify. In these fields, high accuracy is required in work and operation, and the binocular loupes are provided with bright clear image quality, in addition to excellent resolution, wide visual diameter, predetermined focal distance and the like. Further, for scaling adjustments of the loupes, adjustable types are also prepared according to use.

Then, particularly, in binocular loupes used in the medical field, because of being involved in life, the vision correction and astigmatism correction by the binocular loupes are required to correctly adapt to vision of practitioner.

However, in conventional binocular loupes, regardless of high accuracy required in manual operation of medical practitioners, since it is not possible to adapt the focal distance of the lens, which corrects vision of far distance or near distance of the practitioner, to the vision of the operator changing during operation, there is the problem that variations occur in sight accuracy due to changes in vision. In other words, regardless of that the vision of a human always changes corresponding to the extent of physical conditions and fatigue, and further changes in the morning and afternoon even on the same day, it is not possible to adapt the conventional binocular loupes to varying vision of a practitioner, and the practitioner has been forced to operate using the binocular loupes in an improper vision state.

Further, corresponding to the type of procedure e.g. procedure in a narrow eyesight range or procedure in a wide eyesight range, the need arises to change a distance between the procedure/operation portion and the binocular loupes, and therefore, a single practitioner needs to beforehand prepare a plurality of types of binocular loupes with different focal distances.

In order to solve such a problem, binocular loupes have conventionally been known where a plurality of types of focus adjustment units with different focal distances is beforehand prepared, and one is selected from among the units, and can be detachably mounted on the eyepiece unit (for example, see Patent Document 1).

### PRIOR ART DOCUMENT

### Patent Document

Patent Document 1: Japanese Patent Gazette No. 5032332

### DISCLOSURE OF INVENTION

### Problems to be Solved by the Invention

As in Patent Document 1, in the case where a plurality of focus adjustment units is beforehand provided, and one provided with an adjustment lens having matched power is selected from among the units and is used, such a case is advantageous in cost, as compared with the case of preparing a plurality of binocular loupes.

However, also in the case of such conventional techniques, it is necessary to prepare focus adjustment units for each of adjustment lenses with different levels of power, and the effect is restrictive in terms of cost reduction. Further, since the focus adjustment unit is comprised of an independent unit, in mounting the unit on the binocular loupes, it is necessary to use a coupling member to connect between both units, and time and effort is required to mount.

The present invention was made in view of the above-mentioned problem, and it is an object to provide binocular loupes capable of ensuring an optimal focal distance for a wearer, using a simple operation such that a focus adjustment lens is attached and detached.

### Means for Solving the Problem

In order to solve the above-mentioned problem, the present invention is characterized in that binocular loupes having an optical system to enlarge an object on hand to look is provided with a pair of loupe bodies, and focus adjustment units disposed in eyepiece units of the loupe bodies, the focus adjustment units are comprised of focus adjustment lenses having magnetic bodies at the periphery edge drawn to magnets, and magnet rings into which the focus adjustment lenses are fitted, and that the focus adjustment lenses are held detachably by the loupe bodies by attraction between the magnet rings and the magnetic bodies.

In one Embodiment, the magnet ring is fitted into the loupe body to come into with the inner peripheral edge of the eyepiece unit of the loupe body, is nipped by a holding ring having substantially the same diameter as the magnet ring and the eyepiece unit, and is held inside the loupe body.

In another Embodiment, the invention is characterized in that each of the loupes is provided with a holding ring that has an inside diameter substantially equal to an outside diameter of the focus adjustment lens and that is disposed to come into contact with the inner peripheral edge of the eyepiece unit, the magnet ring is provided with a lens receiving unit having an inside diameter substantially equal to the outside diameter of the focus adjustment lens, and a press unit having an inside diameter smaller than the outside diameter of the focus adjustment lens, and that the lens receiving unit cooperates with the holding ring fitted into the loupe body to come into contact with the peripheral edge of the eyepiece unit to hold the focus adjustment lens.

It is possible to form the magnetic body using a ring made of metal into which the focus adjustment lens is fitted.

Further, it is also possible to form the magnetic body using a thin film formed by applying or spraying fine particles of magnetic material with small particle diameters to the surface, or a thin film of magnetic material formed by sputtering and deposition.

### Effect of the Invention

According to the binocular loupes of the present invention, using a simple operation for inserting the focus adjustment lens in the loupe body, it is possible to ensure a focal distance adapted to a wearer at the time with ease. Accordingly, the need is eliminated to provide a plurality of focus adjustment units corresponding to the focal distance, and inexpensive binocular loupes are provided.

### BRIEF DESCRIPTION OF DRAWINGS

FIG. 1 illustrates an entire configuration view of binocular loupes according to an Embodiment of the present invention.

FIG. 2 illustrates a schematic view of a configuration of an optical system of the binocular loupes according to the Embodiment of the present invention.

FIG. 3 illustrates an explanatory view of a state of performing operation with the binocular loupes worn.

FIG. 4 illustrates an explanatory view about a downward mounting angle r in attaching the loupes to carrier lenses.

FIG. 5 illustrates an explanatory view about inside mounting angles p and q in attaching the loupes to the carrier lenses.

FIG. 6 illustrates an explanatory view, looking at the loupes mounted on the carrier lenses from the eyepiece side.

FIGS. 7A and 7B illustrate cross-sectional side views of a part of the eyepiece side of a loupe body and a focus adjustment unit of one Embodiment, where FIG. 7A illustrates the focus adjustment unit in a disassembled state, and FIG. 7B illustrates a state in which the focus adjustment unit is assembled into the eyepiece side of the loupe body.

FIGS. 8A and 8B illustrate cross-sectional side views of a part of the eyepiece side of the loupe body and a focus adjustment unit of another Embodiment, where FIG. 8A illustrates the focus adjustment unit in a disassembled state, and FIG. 8B illustrates a state in which the focus adjustment unit is assembled into the eyepiece side of the loupe body.

FIG. 9 illustrates an entire configuration view of a different type of binocular loupes to apply the present invention.

### BEST MODE FOR CARRYING OUT THE INVENTION

Binocular loupes are configured by attaching each of loupe bodies for both left and right eyes to a glasses frame or head band. Then, the binocular loupes with the loupe bodies attached to the glasses frame have two types including one type of directly supporting by the glasses frame and another type of fitting into holes cut in the glasses lenses (carrier lenses) to attach.

The present invention is applicable to any of binocular loupes described above, and the binocular loupes configured by attaching the loupes to carrier lenses will be described below in detail with reference to drawings.

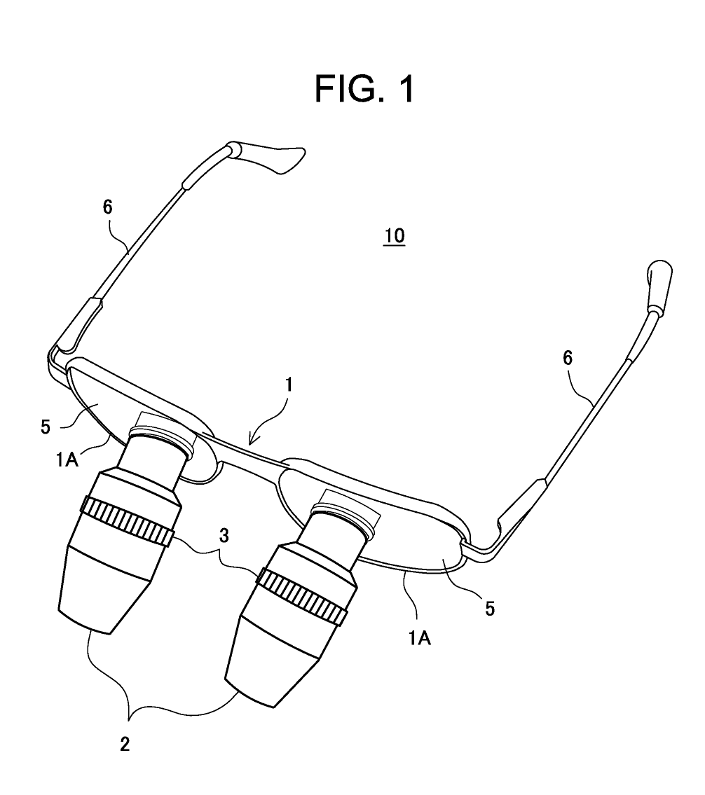

Binocular loupes 10 shown in FIG. 1 are provided with a glasses frame 1, loupe bodies 2 that correspond to both left and right eyes to enlarge an image targeted for operation, and left and right carrier lenses 5 to attach the loupe bodies 2.

The glasses frame 1 has substantially the same structure as normal glasses, and is comprised of rims 1A into which the carrier lenses 5 are fitted, and temple portions 6 put on ears of an observer. Used as materials forming the glasses frame 1 and temple portions 6 are metal such as titanium, synthetic resin and the like hard to rust with flexibility.

Further, materials forming the carrier lenses 5 respectively fitted into rims 1A for both eyes do not need to be always transparent, but are preferably transparent to widen the eyesight in the hand direction of the observer. Further, in the case of requiring correction of vision, corrective lenses are used, and in the case of no need of correction of vision, the lenses may be simple transparent glass. Materials of the lens in this case are glass or plastic.

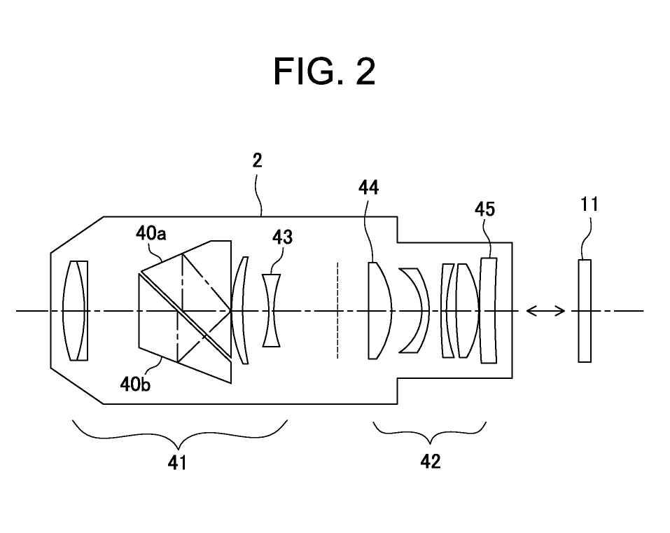

As shown in FIG. 2, in the loupe body 2, an objective lens group 41 and eyepiece group 42 are disposed inside a lens tube along a center axis (optical axis) of the tube. In the objective lens group 41, in order not to decrease image quality in increasing magnification, prisms 40a, 40b are disposed to refract light to be incident. Then, corrective lenses (prescription lenses) 45 to correct vision of a user of the binocular loupes 10 are disposed in end portions on the eyepiece side of the loupe bodies 2, and in no need of correcting vision, as a substitute for the corrective lenses 45, simple glass lenses are disposed to protect the optical system inside the loupe bodies 2.

Although not shown in the figure in detail, it is configured to enable the angle of view of the loupe body 2 to be adjusted, for example, in a range of 3.3 to 4.8 magnifications, by rotating each ring 3 (FIG. 1) provided in the outer region of the loupe body 2 to shift each of a lens 43 of the objective lens group 41 and a lens 44 of the eyepiece group 42 in the optical axis direction. In addition, in the loupe body 2, it is possible to also use the body where distances between lenses are all fixed and the angle of view is a certain magnification.

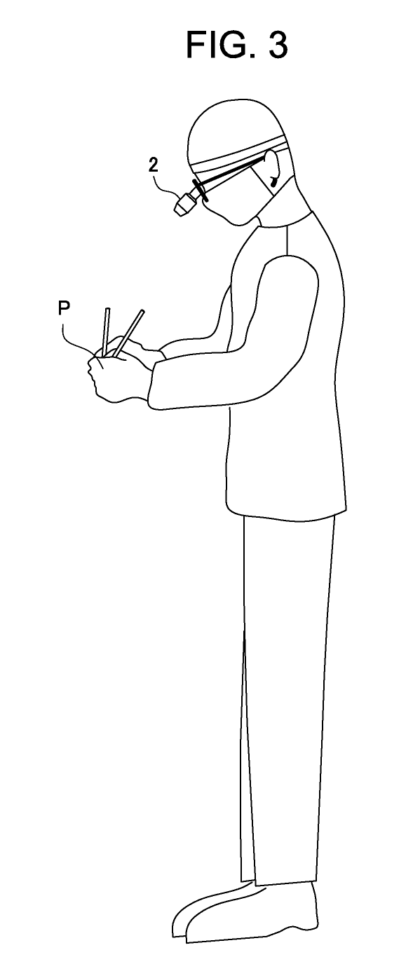

Such binocular loupes 10 are worn on the face with the temple portions 6 put on ears of the user. As shown in FIG. 3, in the case of using when a surgeon performs surgery, the surgeon enlarges and observes an object in a surgical portion P at the front end of surgical instruments in hands through the left and right loupe bodies 2, and at this point, the surgeon drops the line of sight so as to peep through the left and right loupe bodies 2, and stares at the surgical portion P.

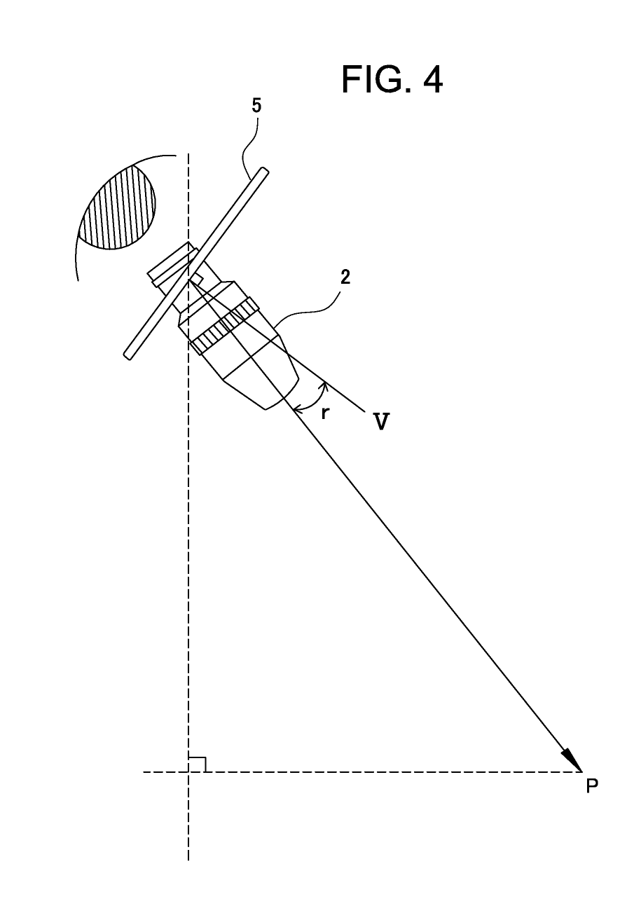

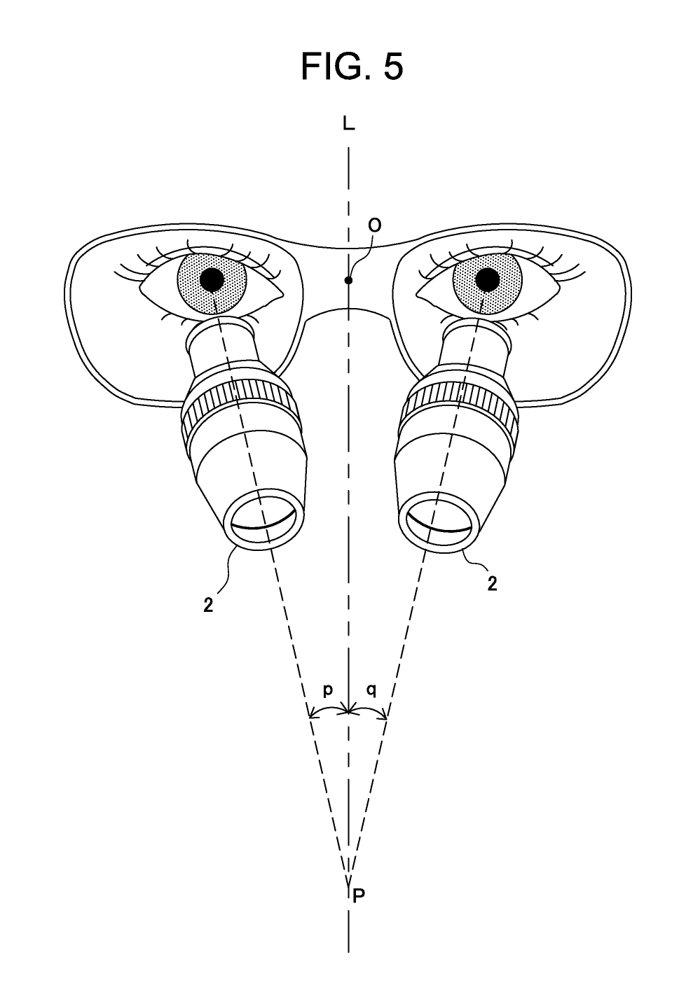

Accordingly, as shown in FIG. 4, each of the left and right loupe bodies 2 is attached, while tilting with respect to an upright line V perpendicular to the plane of the carrier lens 5. The loupe is attached so that the tilt angle is to tilt at a downward angle r (downward mounting angle) with respect to the upright line V. Further, in the horizontal direction, as shown in FIG. 5, the left and right loupe bodies 2 are attached to respectively tilt at angle p and angle q (inside mounting angle) toward a center line L connecting between the center O of the glasses frame 1 and the surgical portion P.

Thus, when the loupe bodies 2 are fitted into holes cut in the carrier lenses 5 and mounted, the bodies are fixed to the carrier lenses 5 in a state of keeping the tilt of the downward mounting angle r and inside mounting angles p, q. Thus, corresponding to an individual user, the downward mounting angle r and inside mounting angles p, q are prescribed to manufacture the binocular loupes 10, and the user adjusts the magnification of the loupe bodies 2, and thereby enlarges an observation target portion to be easy to see.

The downward mounting angle r and inside mounting angles p, q are different corresponding to postures that the user of the binocular loupes 10 takes in operation and features such as a pupillary distance (PD) and the like, and are measured in advance. As a measurement method in this case, there are various kinds of methods such that a user actually reproduces operation postures to perform actual measurement on the angles with a measure and the like, and Japanese Patent Gazettes No. 5652973 and 5311601 disclose a method of measuring based on images obtained by shooting operation postures of an operator.

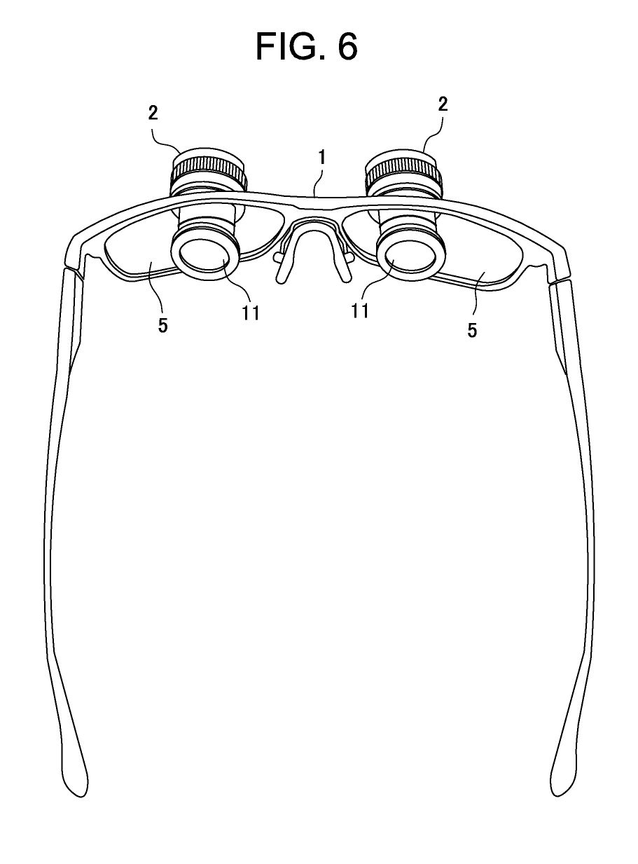

The corrective lenses 45 are disposed in the binocular loupes 10, thereby correct vision of far distance or near distance of a user, and since vision varies corresponding to physical conditions or environment, do not adapt sometimes. Accordingly, when the corrective lenses 45 do temporarily not adapt, as shown in FIG. 6, the user mounts focus adjustment lenses 11 on the eyepiece side of the loupe bodies 2, and the focal distance is thereby adapted to the varied vision. On the other hand, also with respect to a user who does not need correction of vision usually and forms the lenses 45 with glass lenses, when vision varies, the user uses the focus adjustment lenses 11 to adapt the focal distance.

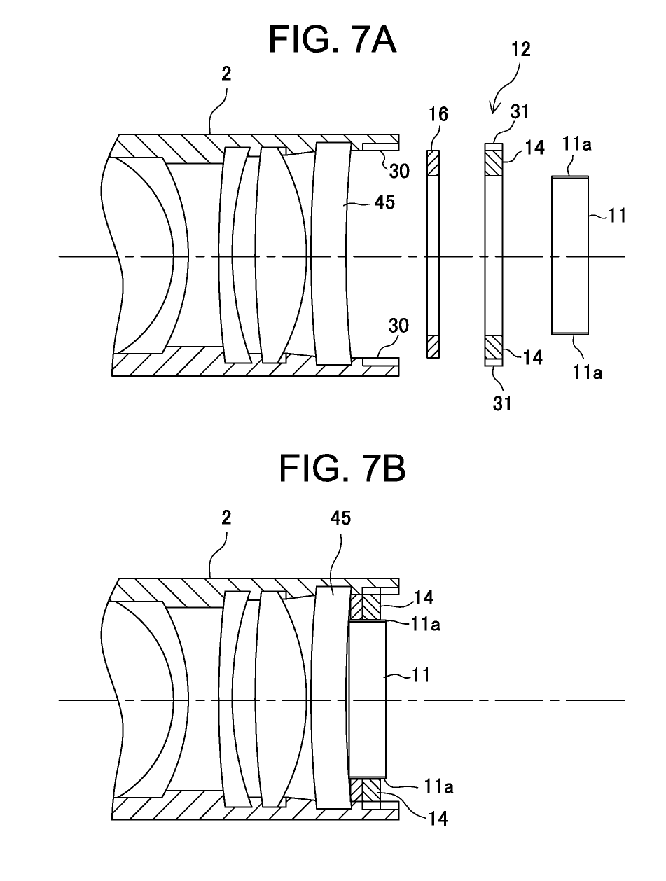

The focus adjustment lens 11 is mounted on the eyepiece unit of the loupe body 2 by the focus adjustment unit 12. FIGS. 7A and 7B illustrate a configuration of the loupe body 2 and focus adjustment unit 12, and each focus adjustment unit 12 is provided with the focus adjustment lens 11, holding ring 14, and magnet ring 16 into which the focus adjustment lens 11 is fitted.

In the focus adjustment lens 11, a magnetic body 11a is formed at the peripheral edge. The magnetic body 11a is formed by applying or spraying fine particles of magnetic material with small particle diameters to the surface at the peripheral edge of the magnet ring 16 to magnetize. Alternatively, a thin film of magnetic material may be formed on the surface by sputtering, deposition and the like. Used as the magnetic material are iron oxide, chromium oxide, cobalt, ferrite, neodymium and the like which do not contain impurities.

The magnetic body 11a may be formed using a ring of magnetic body such as stainless. In this case, the focus adjustment lens 11 is fitted and fixed into an annular ring of the magnetic body 11a of the ring. In addition, in order to protect the outside surface of the magnetic body 11a, it is preferable to apply surface treatment by epoxy coat and the like.

The magnet ring 16 is a ring having an inside diameter substantially equal to an outside diameter of the focus adjustment lens 11 provided with the magnetic body 11a at the peripheral edge, and an outside diameter thereof is substantially equal to an inside diameter of the lens tube of the loupe body 2. Accordingly, the magnet ring 16 is inserted into the lens tube of the loupe body 2.

The holding ring 14 is made of metal, and is drawn by the magnet ring 16. Then, as the magnet ring 16, the ring 14 is formed of a ring having an inside diameter substantially equal to the outside diameter of the focus adjustment lens 11. Further, at the peripheral edge of the holding ring 14 is formed a male screw 31 engaging in a female screw 30 cut in an inner circumferential wall on the opining side of the lens tube of the loupe body 2.

Accordingly, in placing the focus adjustment unit 12 in the eyepiece unit of the loupe body 2, as shown in FIG. 7A, first, the magnet ring 16 is inserted from the opening on the eyepiece side of the loupe body 2 to come into contact with the corrective lens 45, and next, the holding ring 14 is inserted from the opening, while rotating the screw. By this means, the magnet ring 16 is fixed in a state brought into contact with the corrective lens 45.

Next, the focus adjustment lens 11 is inserted into the lens tube of the loupe body 2, and in this case, the inside diameter of each of the magnet ring 16 and the holding ring 14 matches with the outside diameter of the focus adjustment lens 11. Therefore, as shown in FIG. 7B, the user is capable of shifting the focus adjustment lens 11 inside annular rings of the rings 14, 16, until the lens 11 comes into contact with the corrective lens 45. Then, the focus adjustment lens 11 is held inside the loupe body 2 by attraction of the magnet ring 16 and the magnetic body 11a. Accordingly, the user peers through the focus adjustment lens 11, and is thereby capable of observing an image of the object for observation through the optical system of the loupe body 2.

The holding ring 14 is made of metal drawn to the magnet ring 16, and connection between the focus adjustment unit 12 and the loupe body 2 is thereby reliable.

As described above, in the focus adjustment unit 12, the magnet ring 16 and holding ring 14 are fixed and attached inside the loupe body 2, and only the focus adjustment lens 11 shifts inside the annular rings of the magnet ring 16 and holding ring 14, and is detachable from the loupe body 2. Accordingly, a plurality of focus adjustment lenses 11 with different levels of power to compensate for a change in vision is prepared as attachment accessories of the binocular loupes 10, and when the focal distance of the loupe body 2 is not matched, the user selects the focus adjustment lens 11 most suitable for near sight and far sight corresponding to the vision at the time, inserts into the loupe body 2, and is thereby capable of correcting the vision with ease.

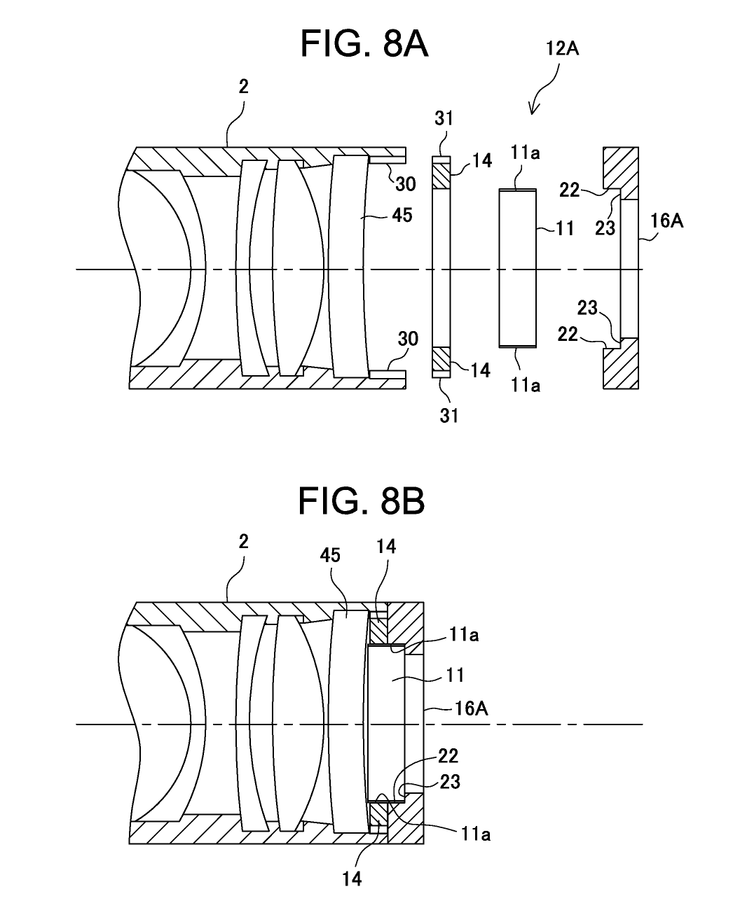

Another Embodiment of the focus adjustment unit will be described. A focus adjustment unit 12A in FIGS. 8A and 8B is provided with the focus adjustment lens 11 and holding ring 14 that are the same in the focus adjustment unit 12. Accordingly, the focus adjustment lens 11 has the magnetic body 11a at the peripheral edge drawn to the magnet, and at the peripheral edge of the holding ring 14 is formed the male screw 31 engaging in the female screw 30 cut in the inner circumferential wall on the opining side of the lens tube of the loupe body 2.

However, in the case of the focus adjustment unit 12A, a magnet ring 16A is different in shape from the magnet ring 16 of the focus adjustment unit 12, and is of two-stage configuration having a lens receiving unit 22 having an inside diameter substantially equal to the outside diameter of the focus adjustment lens 11 and a press unit 23 having an inside diameter smaller than the outside diameter of the focus adjustment lens 11.

Then, in placing the focus adjustment unit 12A in the eyepiece unit of the loupe body 2, as shown in FIG. 8A, first, the holding ring 14 is inserted from the opening on the eyepiece side of the loupe body 2, while rotating the screw, and next, the focus adjustment lens 11 is fitted and shifted into the annular ring of the holding ring 14 to come into contact with the corrective lens 45. By this means, the focus adjustment lens 11 is held by the holding ring 14 fixed threadably.

Next, the magnet ring 16A is brought into contact with the eyepiece unit of the loupe body 2, so that the focus adjustment lens 11 is stored inside the lens receiving unit 22. Accordingly, in the focus adjustment lens 11, the magnetic body 11a is drawn to the magnet ring 16A and holding ring 14, is pressed against the holding ring 14 by the press unit 23 of the magnet ring 16A, and is held inside the loupe body 2. The user peers through the focus adjustment lens 11 from the opening of the magnet ring 16A, and observes an image of the object for observation through the optical system of the loupe body 2.

Accordingly, in the focus adjustment unit 12A, an optimal focus adjustment lens 11 is selected from among focus adjustment lenses 11 with different levels of power beforehand prepared to compensate for a change in vision, and is inserted into the loupe body 2, the inserted focus adjustment lens 11 is mounted on the magnet ring 16A to be stored in the lens receiving unit 22, and the user is thereby capable of correcting the vision.

Then, as in the case of the focus adjustment unit 12, also in the focus adjustment unit 12A, the holding ring 14 is made of metal drawn to the magnet ring 16, and connection between the focus adjustment unit 12A and the loupe body 2 is thereby reliable.

As described above in detail, in the focus adjustment units 12, 12A according to the present invention, various focus corrective lenses 11 with different focal distances are prepared to correct vision of far distance or near distance, and when a wearer of the binocular loupes 10 wants to change to a proper depth of focus corresponding to a state of vision and an ambient brightness state at the time, or corresponding to operation performed in a seated position or standing position, the wearer selects the focus adjustment lens 11 adapted to vision at the time, and mounts on the eyepiece unit of the loupe body 2. Accordingly, it is possible to provide the binocular loupes 10 capable of adjusting to an optimal focal distance, only by mounting the focus adjustment lens 11.

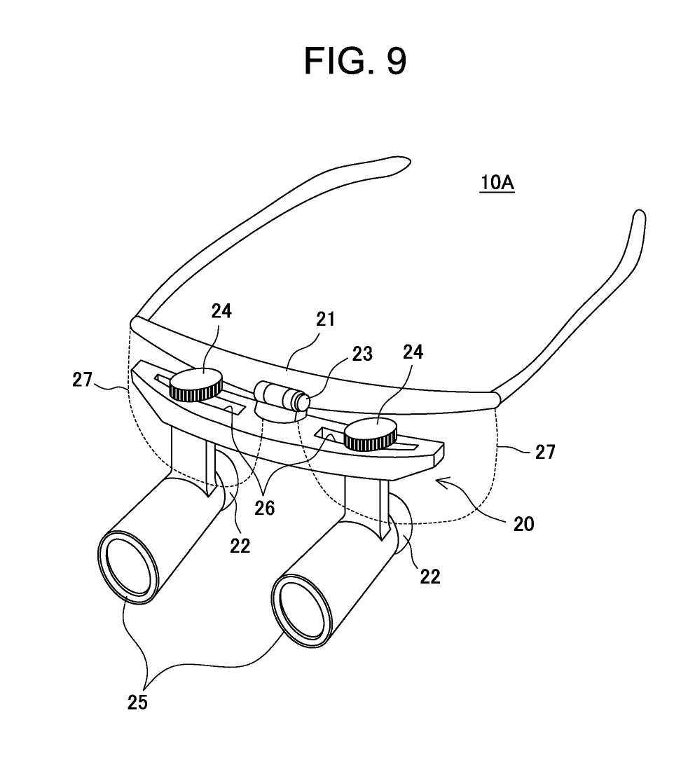

As described previously, the present invention is applicable to binocular loupes without having the carrier lenses, and FIG. 9 shows one example of this type of binocular loupes. The binocular loupes 10A have structure of hanging a loupe body 20 having a pair of left and right loupe bodies 25 on a glasses frame 21 with a rotatable shaft 23. Then, by adjusting screws 24, the loupe body 20 enables the loupe bodies 25 to shift to a position in the vertical direction and leftward/rightward inside slits 26 corresponding to a pupillary distance of an observer. Further, the loupe bodies 25 are supported rotatably by shafts in the perpendicular direction of the screws 24, and it is possible to also adjust the inside mounting angles. Then, the downward mounting angle of the loupe bodies 25 is adjustable by rotating about the shaft 23 as an axis.

Then, the loupe body 25 enables any of the focus adjustment units 12, 12A including the focus adjustment lens 11 to be placed in the eyepiece unit. In addition, this type of binocular loupes includes a type of attaching corrective lenses 27 to the glasses frame without providing the corrective lenses 45 as shown in FIG. 2, and in this case, an observer peers into the loupe bodies 25 through the focus adjustment lenses 11 over the corrective lenses 27.

In the type of binocular loupes 10A thus without mounting the loupes on the carrier lenses, the pupillary distance, inside mounting angle and downward mounting angle are not fixed, and are adjustable arbitrarily. Further, instead of the glasses frame 21, the loupe body 20 may be configured to be hanged on a head band to support.

### INDUSTRIAL APPLICABILITY

The present invention relates to binocular loupes used in medical operations and precision operation which are binocular loupes capable of easily adjusting to the focal distance corresponding to varying vision of a user, and has industrial applicability.

### DESCRIPTION OF THE SYMBOLS

1 Glasses frame
2 Loupe body
10 Binocular loupes
10A Binocular loupes
11 Focus adjustment lens
12 Focus adjustment unit
12A Focus adjustment unit
14 Holding ring
16 Magnet ring
21 Glasses frame

1. Binocular loupes that are binocular loupes having an optical system to enlarge an object on hand to look, characterized by being provided with a pair of loupe bodies, and focus adjustment units disposed in eyepiece units of the loupe bodies,
where the focus adjustment units are comprised of focus adjustment lenses having magnetic bodies at the periphery edge drawn to magnets, and magnet rings into which the focus adjustment lenses are fitted, and the focus adjustment lenses are held detachably by the loupe bodies by attraction between the magnet rings and the magnetic bodies.

2. The binocular loupes as described in claim 1, characterized in that the magnet ring is fitted into the loupe body to come into with the inner peripheral edge of the eyepiece unit of the loupe body, is nipped by a holding ring having substantially the same diameter as the magnet ring and the eyepiece unit, and is held inside the loupe body.

3. The binocular loupes as described in claim 1, characterized by being provided with holding rings each of which has an inside diameter substantially equal to an outside diameter of the focus adjustment lens and is disposed to come into contact with the inner peripheral edge of the eyepiece unit,
where each of the magnet rings is provided with a lens receiving unit having an inside diameter substantially equal to the outside diameter of the focus adjustment lens, and a press unit having an inside diameter smaller than the outside diameter of the focus adjustment lens, and the lens receiving unit cooperates with the holding ring fitted into the loupe body to come into contact with the peripheral edge of the eyepiece unit to hold the focus adjustment lens.

4. The binocular loupes as described in claim 1, characterized in that the magnetic body is a ring made of metal into which the focus adjustment lens is fitted.

5. The binocular loupes as described in claim 1, characterized in that the magnetic body is formed by applying or spraying fine particles of magnetic material with small particle diameters to the surface.

6. The binocular loupes as described in claim 1, characterized in that the magnetic body is a thin film of magnetic material formed by sputtering and deposition.
